(19) United States
(12) Patent Application Publication
(10) Pub. No.: US 2007/0248657 A1
(43) Pub. Date: Oct. 25, 2007

(54) Multi-compartment transdermal pain control device

(76) Inventors: David J. Smith, San Diego, CA (US); Mark J. Badria, San Diego, CA (US); Ollen Williams JR., El Cajon, CA (US); Ludwig J. Weimann, Burlington, VT (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/410,745

(22) Filed: Apr. 25, 2006

(51) Int. Cl. A61K 31/485; A61K 31/445; A61K 9/70

(52) U.S. Cl. 424/449; 514/282; 514/317

(57) ABSTRACT

Multi-compartment patches containing skin-permeable forms of pharmaceutically effective amounts of an opioid agonist, an NMDA receptor antagonist and an anti-inflammatory are useful for the transdermal delivery of the active ingredients to alleviate pain.

### RELATED APPLICATION INFORMATION

This application is related to U.S. application Ser. No. 11/097,878, filed Apr. 1, 2005, which is hereby incorporated by reference in its entirety.

### BACKGROUND OF THE INVENTION

1. Field of the Invention

This invention relates to compositions useful for alleviating pain and methods for their delivery to humans. More particularly, this invention relates to compositions comprising an opioid agonist, an NMDA receptor antagonist, and an anti-inflammatory, and methods for the transdermal delivery of those compositions to relieve pain.

2. Description of the Related Art

The treatment of physical pain concerns health care professionals throughout the world. The treatment of chronic pain is particularly challenging because of the frequent need for repeated administration of pain relief medication. Chronic pain is generally considered to be pain that continues a month or more beyond the usual recovery period for an illness or injury or pain that goes on over months or years as a result of a chronic condition. It may be continuous or come and go. It is estimated that chronic pain disables, to some degree, about 86 million Americans. It is regarded as a source of frustration for the health care professionals who care for the patient, and affects the quality of life and economic security not only of the person with pain, but also his or her family. It is estimated that United States business and industry loses about $90 billion annually to sick time, reduced productivity, and direct medical and other benefit costs due to chronic pain among employees. In some cases, repeated administration of the pain relief medication causes sufferers of chronic pain to develop an undesirable tolerance or addiction, creating further health issues for the patient and additional challenges for the health care professional.

There are a number of methods for administering pain relief medications, including oral and parenteral (administered in a manner other than through the digestive tract). Oral administration is most frequently accomplished by formulating the pain relief medication into tablet or syrup and allowing the patient to swallow it. This method is simple, well accepted and relatively painless, but may be problematic for uncooperative patients. Also, there is often a considerable lapse of time between administration of the pain relief medication and its therapeutic effect because of the time needed for gastrointestinal absorption. This time lag is of particular concern when a patient is suffering from severe or chronic pain. Faster administration may be accomplished by direct injection of the pain relief medication, but most people consider the injection itself to be painful and thus undesirable. A transdermal delivery patch for the delivery of fentanyl has been commercialized (DURAGESIC®, Ortho-McNeil). However, existing transdermal delivery systems are not entirely satisfactory for the transdermal delivery of multi-component pain formulations.

### SUMMARY OF THE INVENTION

This invention is directed to transdermal pain relief compositions, delivery systems and methods. Preferred embodiments are directed to multi-compartment transdermal delivery patches that contain an opioid agonist, an N-methyl-D-aspartate receptor antagonist and an anti-inflammatory, and methods of using such patches for the relief of pain. The opioid agonist, N-methyl-D-aspartate receptor antagonist and anti-inflammatory may be referred to herein (each individually or collectively) as the active ingredients of the multi-compartment transdermal delivery patch.

Opioid agonists are preferred pain relief drugs. To inhibit the development of tolerance and/or addiction to the opioid agonists by the patient, preferred multi-compartment transdermal delivery patches also contain a substance that blocks the N-methyl-D-aspartate receptor, herein referred to as an “NMDA receptor antagonist.” Preferred multi-compartment transdermal delivery patches also contain an anti-inflammatory drug, preferably a non-steroidal anti-inflammatory drug (NSAID). Contrary to the disclosure of U.S. Patent Application Publication 2003/0199439 A1, preferred drug combinations need not contain a α3β4 nicotinic receptor antagonist. Thus, preferred multi-compartment transdermal delivery patches do not contain a pharmaceutically effective amount of a α3β4 nicotinic receptor antagonist.

It has been found that substantially simultaneous transdermal delivery of the active ingredients (an opioid agonist, an NMDA receptor antagonist and an anti-inflammatory) is facilitated by using a multi-compartment transdermal delivery patch in which at least a portion of the NMDA receptor antagonist and the anti-inflammatory are in separate compartments. It has also been found that transdermal delivery of the active ingredients is facilitated where the pH of the contents of at least two of the compartments are different from one another. For example, in an embodiment in which at least a portion of the NMDA receptor antagonist and the anti-inflammatory are in separate compartments of the multi-compartment patch, the pH in the compartment that contains the NMDA receptor antagonist is preferably a basic pH (higher than 7.0, e.g., about 8 to about 12), and the pH in the compartment that contains the anti-inflammatory is preferably an acidic pH (lower than 7.0, e.g., about 3 to about 6).

Surprisingly, it has been found that the transdermal flux of the opioid agonist is facilitated by the presence of the NMDA receptor antagonist. In an embodiment, at least a portion of the opioid agonist and the NMDA receptor antagonist are in the same compartment. The amount of NMDA antagonist is preferably an amount that is effective to increase transdermal flux of the opioid agonist. The compartments may contain various opioid agonists, NMDA receptor antagonists and anti-inflammatories. For example, the opioid agonist can be fentanyl and/or sufentanil, the NMDA receptor antagonist can be dextromethorphan, and the anti-inflammatory can be ketorolac. One or more of the compartments may contain additional ingredients such as a skin permeation enhancer. Linoleic acid is an example of a preferred skin permeation enhancer, and has been found to be particularly useful for enhancing the transdermal flux of the anti-inflammatory.

An embodiment provides a multi-compartment transdermal delivery patch that contains pharmaceutically effective amounts of skin-permeable forms of an opioid agonist, a NMDA receptor antagonist different from the opioid agonist, and an anti-inflammatory different from the opioid agonist and different from the NMDA receptor antagonist. In this embodiment, at least a portion of the NMDA receptor antagonist and the anti-inflammatory are in separate compartments. For example, in an embodiment, a first compartment contains a first pharmaceutical composition and a second compartment contains a second pharmaceutical composition. In such an embodiment, the first pharmaceutical composition contains the opioid agonist and the NMDA receptor antagonist, and the second pharmaceutical composition contains the anti-inflammatory.

Another embodiment provides a multi-compartment transdermal delivery patch that contains a first compartment, a second compartment and a third compartment. The first compartment contains a pharmaceutically effective amount of a skin-permeable form of an opioid agonist. The second compartment contains a pharmaceutically effective amount of a skin-permeable form of an NMDA receptor antagonist different from the opioid agonist. The third compartment contains a pharmaceutically effective amount of a skin-permeable form of an anti-inflammatory different from the opioid agonist and different from the NMDA receptor antagonist. Preferably, the opioid agonist is at a pH in the range of about 8 to about 12, the NMDA receptor antagonist is at a pH in the range of about 8 to about 12, and/or the anti-inflammatory is at a pH in the range of about 3 to about 6.

Other embodiments provide methods of treating pain that include applying a multi-compartment transdermal delivery patch as described herein to a human suffering from pain for a period of time effective to at least partially relieve the pain.

These and other embodiments are described in greater detail below.

### BRIEF DESCRIPTION OF THE DRAWINGS

These and other aspects of the invention will be readily apparent from the following description and from the appended drawings (not to scale), which are meant to illustrate and not to limit the invention, and in which:

FIGS. 1A and 1B are top and cross-sectional schematic views, respectively, of a multi-compartment transdermal delivery patch having two compartments. FIG. 1C is a top view schematic view of a multi-compartment transdermal delivery patch having three compartments.

FIG. 2 is a plot illustrating the flux of fentanyl, ketorolac tromethamine and dextromethorphan in 100% ethanol from a single compartment transdermal delivery patch through human cadaver epidermis as a function of time.

FIG. 3 is a plot illustrating the flux of fentanyl, ketorolac tromethamine and dextromethorphan through human cadaver epidermis as a function of time at a pH of 5.6 in 80/20 ethanol/water.

FIG. 4 is a plot illustrating the flux of fentanyl, ketorolac tromethamine and dextromethorphan through human cadaver epidermis as a function of time at a pH of 7.3 in 80/20 ethanol/water.

FIG. 5 is a plot illustrating the flux of fentanyl, ketorolac tromethamine and dextromethorphan through human cadaver epidermis as a function of time at a pH of 8.6 in 80/20 ethanol/water.

FIG. 6 is a plot illustrating the flux of fentanyl and dextromethorphan from a fentanyl/dextromethorphan mixture in 80/20 ethanol/water at pH of 9.4 through human cadaver epidermis as a function of time, as compared to the flux of fentanyl from a commercially available DURAGESIC® fentanyl patch.

FIG. 7 is a plot illustrating the flux of fentanyl, ketorolac tromethamine and dextromethorphan from a multi-compartment transdermal delivery patch through human cadaver epidermis as a function of time.

### DETAILED DESCRIPTION OF THE PREFERRED EMBODIMENTS

An embodiment provides a multi-compartment transdermal delivery patch that contains pharmaceutically effective amounts of (a) an opioid agonist; (b) an NMDA receptor antagonist different from the opioid agonist; and (c) an anti-inflammatory, the anti-inflammatory being different from the opioid agonist and different from the NMDA receptor antagonist. To facilitate transdermal administration, the opioid agonist, the NMDA receptor antagonist and the anti-inflammatory are preferably each in a skin-permeable form. The term “pain” is used herein to refer to the condition to which the patient is subject and thus includes associated inflammation. It will be understood that a patient's pain is commonly, and in fact usually, associated with and resulting from inflammation at the site of the dysfunction, trauma, chronic disease or the like.

The term “opioid agonist” is used herein in the ordinary sense and thus includes opiates, opiate derivatives, opioids, and other substances whose effects are mediated by the same receptor, including mixtures thereof. Non-limiting examples of suitable opioid agonists include: alfenanil; allylprodine; alphaprodine; anileridine; benzitramide; benzylmorphine; beta-endorphin; buprenorphine; butorphanol; carfentanil; clonitazene; codeine; cyclazocine; cyclozine, desomorphine; dextromoramide; dezocine; diamorphine; diampromide; dihydromorphine; dimenoxadol; fentanyl; sufentanil; lofentanil; morphine; normorphine; dihydrocodeine; levorphanol; oxycodone; oxycodone; propoxyphene; meperidine; methadone; normethadone; meptazinol; nicomorphine; pentazocine, remifentanil, heroin, morphine-6-glucuronide; nalbuphine; meptazinol; pethidine; hydromorphone; piritramide; nicomorphine; tilidine; tramadol; opium; met-enkaphalin; delta-enkephalin; dynorphin A; peptide F; Leu-enkephalin; N-alpha-acetylmethadone; dihydromorphine; etorphine; and oxymorphone. Reference herein to the class of opioid agonists or to a particular opioid agonist will be understood to include reference to pharmaceutically acceptable acids, bases and/or salts thereof, unless the context clearly indicates otherwise. Non-limiting examples of particularly preferred opioid agonists include fentanyl, hydromorphone, hydrocodone, ketamine, methadone, oxycodone, oxymorphone, propoxyphene, and sulfentanil.

NMDA receptor antagonists are substances known to those skilled in the art that block the NMDA receptor or that block a major intracellular consequence of NMDA receptor activation, see, e.g., U.S. Pat. Nos. 5,321,012; 5,654,281 and 5,869,498, all of which are hereby incorporated by reference in their entireties, and particularly for the purpose of describing NMDA receptor antagonists and their uses. The NMDA receptor antagonist may be a mixture. Non-limiting examples of preferred NMDA receptor antagonists include amantadine, amitriptyline, D,L-2-amino-5-phosphono valeric acid, dextromethorphan, ketamine, and methadone. Reference herein to the class of NMDA receptor antagonists or to a particular NMDA receptor antagonist will be understood to include reference to pharmaceutically acceptable acids, bases and/or salts thereof, unless the context clearly indicates otherwise. Since some substances, e.g., ketamine and methadone, may be classified as both opioid agonists and NMDA receptor antagonists, it is understood that the opioid agonist in any particular formulation or multi-compartment transdermal delivery patch is different from the NMDA receptor antagonist.

The term “anti-inflammatory” refers to a broad class of agents useful for reducing and/or preventing inflammation, and thus includes steroidal anti-inflammatories and non-steroidal anti-inflammatories (NSAIDS). The anti-inflammatory may be a mixture. Reference herein to the class of anti-inflammatories or to a particular anti-inflammatory will be understood to include reference to pharmaceutically acceptable acids, bases and/or salts thereof, unless the context clearly indicates otherwise. Non-limiting examples of steroidal anti-inflammatories include corticosteroids such as alcometasone, clocortolone, dexamethasone, hydrocortisone, hydrocortisone 21-acetate, prednisone, hydrocortisone 17-valerate, hydrocortisone 17-butyrate, betamethasone valerate, triamcinolone acetonide, flucinonide, desonide, flucinolone acetonide, dexamethasone, dexamethasone 21-phosphate, prednisolone, prednisolone 21-phosphate, haloprednone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone, flumethasone pivalate, flunisolide acetate, flucortolone, fluorometholone, fluperolone acetate, fluprednisolone, fluprednisolone valerate, meprednisone, methyl prednisolone, paramethasone acetate, prednisolamate, prednival, triamcinolone, triamcinolone hexacetonide, cortivazol, formocortal, nivazol, and methylprednisone.

Non-limiting examples of non-steroidal anti-inflammatories include diclodenac, diflunisal, fenoprofen, flurbiprofen, ibuprofen, indomethacin, ketoprofen, melcofenamate, mefenamic acid, naproxen, phenylbutazone, piroxicam, sulindac, tiaprofenic acid, alcolfenac, desoxysulindac, aspirin, salicylamide, salicyclic acid, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole neocinchophen, nimazole, proxazole citrate, tesicam, tesimide, tramadol, triflumidate, and tolmetin. Non-limiting examples of preferred non-steroidal anti-inflammatories include ketorolac, ibuprofen, nabumetone, diclofenac, etodolac, and piroxicam.

As noted above, the opioid agonist, NMDA receptor antagonist and anti-inflammatory may be referred to herein (each individually or collectively) as the active ingredients of the multi-compartment transdermal delivery patch. As discussed in greater detail below and illustrated in the examples, it has been found that the pH of the active ingredients (and/or the formulations into which they are incorporated) affects skin permeability. Preferably, at least a portion of the NMDA receptor antagonist and the anti-inflammatory are placed into separate compartments of a multi-compartment transdermal delivery patch to permit the pH of each to be separately selected to enhance skin-permeability. In an embodiment, the pH of the NMDA receptor antagonist and/or the formulation into which it is incorporated is basic, preferably in the range of about 8 to about 12, more preferably about 9 to about 11. In an embodiment, the pH of the anti-inflammatory and/or the formulation into which it is incorporated is acidic, preferably in the range of about 3 to about 6, more preferably about 4 to about 5. In an embodiment, the pH of the opioid agonist and/or the formulation into which it is incorporated is basic, preferably in the range of about 8 to about 12, more preferably about 9 to about 11. The pH of the active ingredient formulation may be adjusted by appropriate addition of known pH-adjusting agents, using routine experimentation and the guidance provided by the teachings herein.

In some embodiments, the opioid antagonist and NMDA receptor antagonist are formulated into a single composition having a pH that is basic, preferably in the range of about 8 to about 12, more preferably about 9 to about 11. Since the preferred pH ranges of the opioid agonist and the NMDA receptor antagonist have been found to be compatible, in an embodiment the opioid agonist and the NMDA receptor antagonist are placed into one compartment of a two-compartment transdermal patch (such as the patch 100a described below) and the anti-inflammatory is placed into the other compartment. In another embodiment, the active ingredients are each placed into separate compartments of a three-compartment transdermal patch such as the patch 100b described below.

The active ingredients are typically formulated to facilitate transdermal delivery and are preferably in a skin-permeable form. For example, the active ingredients are typically dissolved in a solvent such as ethanol or aqueous ethanol having a weight ratio of ethanol/water in the range of about 10/90 to about 90/10, preferably in the range of about 50/50 to about 80/20. The active ingredient compositions may be formulated to include a skin permeation enhancer that enhances the penetration of the drugs through the skin. It is believed that skin penetration enhancers facilitate transfer of the drug components through the stratum corneum and into the dermis to provide a local effect. For a discussion of use of penetration enhancers see generally, PERCUTANEOUS PENETRATION ENHANCERS (Eric W. Smith & Howard I. Maibach eds. 1995); Ghosh, T. K. et al. 17 PHARM. TECH. 72 (1993); Ghosh, T. K. et al. 17 PHARM. TECH. 62 (1993); Ghosh, T. K. et al. 17 PHARM. TECH. 68 (1993), all of which are hereby incorporated by reference in their entireties. Preferred penetration enhancers are pharmacologically inert, non-toxic, and non-allergenic, have rapid and reversible onset of action, and are compatible with the active ingredient compositions. Non-limiting examples of penetration enhancers include transcutol P, ethyl alcohol (ethanol), isopropyl alcohol, lauryl alcohol, linoleic acid, salicylic acid, octolyphenylpolyethylene glycol, polyethylene glycol 400, propylene glycol, N-decylmethylsulfoxide, DMSO and the azacyclo compounds, as disclosed in U.S. Pat. Nos. 4,755,535; 4,801,586; 4,808,414; and 4,920,101, all of which are hereby incorporated by reference in their entireties and particularly for the purpose of describing skin penetration enhancers. Preferably, the skin penetration enhancer is ethanol, more preferably linoleic acid. The amount of skin penetration enhancer may be determined by routine experimentation. In an embodiment, the active ingredient formulation includes a penetration enhancer (such as ethanol) that also functions as a solvent for one or more of the active ingredients. In the context of describing the presence, placement, properties or activity of active ingredients in multi-compartment transdermal delivery patches, it will be understood that reference herein to the active ingredients generally or to particular active ingredients includes active ingredient formulations or compositions that contain additional ingredients (such as a solvent and/or skin penetration enhancer), unless clearly stated otherwise. For example, reference herein to a patch that contains fentanyl will be understood to include, e.g., a patch that contains a formulation comprising 80/20 ethanol/water and a pharmaceutically effective amount of fentanyl at a pH of about 8-9 as described elsewhere herein.

The active ingredients are preferably contained in a multi-compartment transdermal delivery patch that is suitable for application to the skin. The multi-compartment transdermal delivery patch preferably comprises at least the active ingredients and a covering layer that permits the patch to be placed on the area of skin to be treated. Preferably, the multi-compartment transdermal delivery patch maximizes drug delivery through the stratum corneum and viable epidermis into the capillary cardiovascular system, reduces lag time, promotes uniform absorption, and/or reduces mechanical rub-off. Preferably, the mechanical patch components conform to the skin during movement to provide comfort and prevent undue shear and delamination.

A variety of multi-compartment transdermal delivery patch technologies are suitable or may be readily adapted for the delivery of the active ingredients, including (1) the matrix-type patch; (2) the reservoir-type patch; (3) the multi-laminate drug-in-adhesive type patch; (4) the monolithic drug-in-adhesive type patch; and (5) hydrogel patch; see generally Ghosh, T. K.; Pfister, W. R.; Yum, S. I. Transdermal and Topical Drug Delivery Systems, Interpharm Press, Inc. p. 249-297, hereby expressly incorporated herein by reference). These patches are well known in the art and various designs are available commercially. The active ingredients can be incorporated into the patch in various ways. For example, the active ingredients may be incorporated into two or three separate adhesive sections of a drug-in-adhesive or hydrogel patch. The multi-compartment drug-in-adhesive patch design is characterized by the inclusion of a skin contacting adhesive layer (containing the active ingredients), a backing film and preferably, a release liner. The adhesive layer is in two or more sections to allow each to contain a different active ingredient formulation. The adhesive functions both to release the active ingredient and to maintain contact between the active ingredient formulation and the skin. Also, drug-in-adhesive type patches are thin and comfortable (see, e.g., U.S. Pat. No. 4,751,087, which is hereby incorporated by reference in its entirety and particularly for the purpose of describing drug-in-adhesive type transdermal delivery patches). Preferred multi-compartment transdermal delivery patches comprising the active ingredients described herein have advantages over conventional methods of administration. One advantage is that the dose is controlled by the patch's surface area. Other advantages may include, in certain embodiments, relatively constant rate of administration, longer duration of action (the ability of to adhere to the skin for 1, 3, 7 days or longer); improved patient compliance, non-invasive dosing, and reversible action (i.e., the patch can simply be removed).

In a preferred embodiment, the active ingredients are contained in a multi-compartment reservoir-type transdermal delivery patch. The reservoir-type patch is typically characterized by a backing film, a reservoir, a drug permeable membrane, a skin contact adhesive and a reservoir compartment comprising the active ingredients (see, e.g., U.S. Pat. No. 4,615,699, which is hereby incorporated by reference in its entirety and particularly for the purpose of describing transdermal delivery patches that comprise a reservoir). Multi-compartment transdermal delivery patches contain multiple reservoirs, e.g., two, three or more reservoirs. The drug-permeable membrane is typically coated with the skin contact adhesive and holds the reservoir(s) adjacent to the skin. The multi-compartment reservoir-type transdermal delivery patch preferably comprises a permeable membrane with a degree of porosity of about 3 to about 30 percent. The volume and surface area of the patch may be adjusted depending on the application. The volume is preferably in the range of about 0.2 mL to about 2.0 mL, and the surface area of the permeable membrane is preferably in the range of from about 5 cm² to about 40 cm². In multi-compartment patches, the adhesive-coated membranes under each reservoir are preferably separated by a spacer or gap of about 0.1 cm to about 0.3 cm in order to slow or prevent the drugs in each of the compartments from mixing within the adhesive prior to transdermal flux.

In an embodiment, an advantage of the multi-compartment reservoir patch is that it provides a way of achieving relatively high transdermal fluxes of each of the drugs from their respective compartments, e.g., from saturated solutions. In an embodiment, an advantage of the multi-compartment reservoir patch is that it provides a way to deliver drugs or forms of drugs that are mutually incompatible from separate reservoirs. In an embodiment, an advantage of the multi-compartment reservoir patch is that it provides a way to deliver drugs or forms of drugs that are macro-incompatible when mixed with adhesives such as in drug-in-adhesive type patches, but which diffuse readily through the adhesive layer in molecular form.

Within each of the active ingredient compositions in the various reservoirs, the concentration of each of the active ingredients (opioid agonist, NMDA receptor antagonist and anti-inflammatory) is typically in the range of about 0.1 percent to about 90 percent, preferably in the range of about 0.25 percent to about 50 percent, by weight based on the total weight of the composition in the reservoir. For any particular reservoir-type patch, the concentration of each of the active ingredients is preferably adjusted to provide the desired dosage to the patient when the patch is applied to the skin, taking into account the porosity of the membrane, the surface area of the patch, the efficiency of the penetration enhancer, and potency of the active ingredient, as determined by routine experimentation. For example, typical amounts of various active ingredient (on a per 10 sq. cm patch basis) are as follows: the amount of fentanyl is preferably in the range of about 1.5 mg to about 15 mg; the amount of sufentanil is preferably in the range of about 0.15 mg to about 1.5 mg; the amount of ketorolac is preferably in the range of about 10 mg to about 180 mg; the amount of dextromethorphan is preferably in the range of about 12 mg to about 36 mg; the amount of dexamethasone is preferably in the range of about 2 mg to about 32 mg; the amount of amantadine is preferably in the range of about 5 mg to about 200 mg; the amount of amitryptiline is preferably in the range of about 30 mg to about 300 mg; the amount of methadone is preferably in the range of about 45 mg to about 180 mg; and the amount of betamethasone is preferably in the range of about 1 mg to about 16 mg.

To facilitate skin permeation, the composition in the reservoir preferably comprises a carrier or solvent in an amount that is effective to dissolve the opioid agonist, NMDA receptor antagonist and anti-inflammatory. In a preferred embodiment, the carrier is a penetration enhancer as described above, more preferably ethanol or aqueous ethanol having a weight ratio of ethanol/water in the range of about 10/90 to about 90/10, preferably in the range of about 50/50 to about 80/20. The amount of carrier in the reservoir is typically adjusted so that the composition in the reservoir has the desired concentration of active components. Typically, the amount of carrier in the reservoir is in the range of about 10% to about 99.9%, by weight based on the total weight of the composition. In an embodiment, the amount of carrier is selected so that the drug is at or near a saturation concentration, to thereby facilitate or maximize the transdermal flux of the drug. The composition in the reservoir may further comprise a pharmaceutically acceptable thickening agent to facilitate handling and reduce leakage. A wide variety of pharmaceutically acceptable thickening agents are known to those skilled in the art. Hydroxyethylcellulose (HEC) is an example of a preferred thickening agent. The amount of thickening agent in the composition is preferably in the range of about 0.2% to about 4%, by weight based on the total weight of the composition in the reservoir.

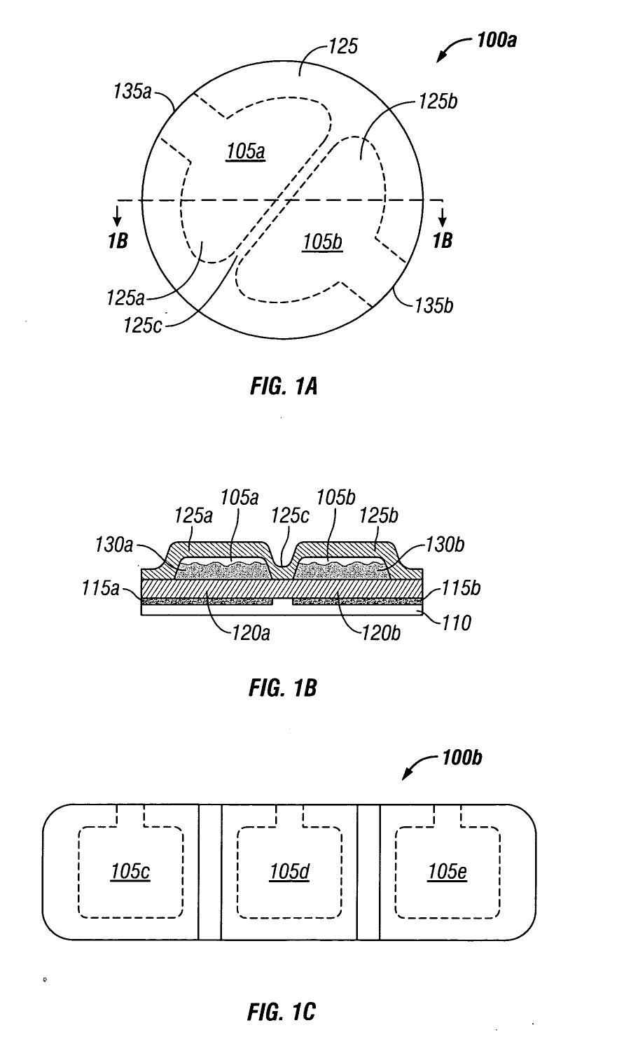

FIGS. 1A-1C illustrate embodiments of multi-compartment transdermal delivery patches that contains pharmaceutically effective amounts of skin-permeable forms of an opioid agonist, an NMDA receptor antagonist, and an anti-inflammatory, where at least a portion of the NMDA receptor antagonist and the anti-inflammatory are in separate compartments. FIGS. 1A and 1B are schematic top and side cross-sectional views, respectively, of a two-compartment patch 100a (containing a first internal compartment 105a and a second internal compartment 105b), and FIG. 1C is a schematic top view of a three-compartment patch 100b (containing a first internal compartment 105c, a second internal compartment 105d, and a third internal compartment 105e). The sizes of the patches 100a-b (and the compartments 105a-e) are generally selected to contain pharmaceutically effective amounts of the active ingredients. For any given patch, the compartments may be substantially equal in size as illustrated, or may be of different sizes.

In the illustrated embodiment, the two-compartment patch 100a comprises a release liner 110 and a skin contact adhesive 115a-b in contact with the release liner 110. A first compartment 105a is separated from the release liner 110 by a drug permeable membrane 120a coated with adhesive layer 115a, and a second compartment 105b is separated from the release liner 110 by a drug permeable membrane 120b coated with an adhesive layer 115b. The compartments 105a and 105b are enclosed by a backing layer 125 that is attached (e.g., heat sealed) to the outer edges of the drug permeable membrane 120a-b. Thus, as illustrated in FIG. 1B, the compartment 105a is bounded by the underlying drug permeable membrane 120a and an overlying portion 125a of the backing layer 125, and the compartment 105b is bounded by the underlying drug permeable membrane 120b and an overlying portion 125b of the backing layer 125. In the illustrated embodiment, a portion of the backing layer 125 is attached (e.g., heat sealed) to a central portion of the drug permeable membrane 120a-b, thereby forming a barrier 125c between the compartments 105a and 105b. FIG. 1B is not to scale and thus, for example, the relative thicknesses of the various layers may differ from what is illustrated.

The multi-compartment transdermal delivery patch 100a contains pharmaceutically effective amounts of skin-permeable forms of an opioid agonist, an NMDA receptor antagonist, and an anti-inflammatory. In the illustrated embodiment, the opioid agonist is fentanyl, the NMDA receptor antagonist is dextromethorphan, and the anti-inflammatory is ketorolac (e.g., ketorolac tromethamine), and these active ingredients are components of pharmaceutical compositions that are formulated so that at least a portion of the NMDA receptor antagonist and the anti-inflammatory are in separate compartments of the patch 100a. In particular, the compartment 105a contains a first pharmaceutical composition 130a that comprises the fentanyl and the dextromethorphan, and the compartment 105b contains a second pharmaceutical composition 130b that comprises the ketorolac tromethamine. In the illustrated embodiment, the first pharmaceutical composition 130a comprises a solvent (80/20 ethanol/water) having a pH of about 9-10 in which the concentration of fentanyl is about 32.5 milligrams per milliliter (mg/mL) and the concentration of dextromethorphan is about 70 mg/mL. The second pharmaceutical composition 130b of the illustrated embodiment comprises a solvent (80/20 ethanol/water) having a pH of about 4-5 in which the concentration of ketorolac tromethamine is about 200 mg/mL. The first and second pharmaceutical compositions 130a-b may be loaded into the patch 100a via syringe using ports 135a-b, respectively.

FIG. 1C illustrates a three-compartment patch 100b that is generally similar in design and construction to the two-compartment patch 100a except that it has three compartments instead of two. In an embodiment, the first compartment 105c contains an opioid agonist formulation, the second compartment 105d contains a NMDA receptor antagonist formulation, and the third compartment 105e contains an anti-inflammatory formulation, each as generally described above. For example, the opioid agonist (comprising, e.g., an aqueous ethanol solution of fentanyl and/or sulfentanil) in the first internal compartment 105c is preferably at a pH in the range of about 8 to about 12; the NMDA receptor antagonist formulation (comprising, e.g., an aqueous ethanol solution of dextromethorphan) in the second internal compartment 105d is preferably at a pH in the range of about 8 to about 12; and the anti-inflammatory formulation (comprising, e.g., an aqueous ethanol solution of ketorolac tromethamine) is preferably at a pH in the range of about 3 to about 6. The configurations of the multi-compartment transdermal delivery patches 100a-b are examples, and other suitable multi-compartment transdermal delivery patch configurations may also be used, as determined by routine experimentation.

The multi-compartment transdermal delivery patches described above may be manufactured, packaged, stored and labeled according to standard procedures. For example, see the procedures described in Bova et al., Product Development and Technology Transfer for Transdermal Therapeutic Systems in TRANSDERMAL CONTROLLED SYSTEMIC MEDICATIONS 379-396 (Y. W. Chien ed. 1987); J. W. Dohner, Development of Processes and Equipment for Rate Controlled Transdermal Therapeutic Systems in TRANSDERMAL CONTROLLED SYSTEMIC MEDICATIONS 349-364 (Y. W. Chien ed. 1987); H-M Wolf et al., Development of Processes and Technology for Adhesive-Type Transdermal Therapeutic Systems in TRANSDERMAL CONTROLLED SYSTEMIC MEDICATIONS 365-378 (Y. W. Chien ed. 1987), all of which are hereby incorporated by reference in their entireties.

An embodiment provides a method for treating pain, comprising applying a multi-compartment transdermal delivery patch as described herein to a human suffering from pain for a period of time effective to at least partially relieve the pain. For example, after removal of the release liner 110, the transdermal patch 100a may be applied to the skin in such a way that the skin contact adhesive 115a-b contacts the skin, thereby holding the drug permeable membranes 120a-b in operable proximity to the skin. During treatment, the first pharmaceutical composition 130a passes through the membrane 120a and the adhesive 115a to the skin, while the second pharmaceutical composition 130b passes through the membrane 120b and the adhesive 115b to the skin. The active ingredients in both the pharmaceutical compositions 130a-b then enter the body transdermally. The dosage to achieve at least partial pain relief is typically determined by the active surface area of the medicated portion of the patch in operable proximity to the skin (e.g., the surface area of drug permeable membranes 120a-b). Several dosage strengths are advantageous depending upon the severity of the pain. In general, a physician can begin dosing with a low or intermediate strength patch and then, depending upon the effectiveness, adjust the dosage up or down by prescribing a patch of higher or lower active concentration or a patch of larger or smaller surface area, or, in some cases, multiple patches. In general, the active ingredient formulations will comprise from about 0.001 percent to about 20 percent by weight of the patch, typically from about 1 percent to about 25 percent by weight of the patch. For matrix (e.g., drug-in-adhesive) type patches, the active ingredient formulations typically comprise from about 0.5 percent to about 20 percent by weight of the patch. For patches comprising a hydrogel, the active ingredient formulations typically comprise from about 0.5 percent to about 10 percent by weight of the patch. Fresh patches may be administered multiple times per day, but, preferably, a fresh patch is administered about every 18 to about every 48 hours, more preferably about every 72 hours. In an embodiment, a pharmaceutical composition in a compartment comprises an amount of an NMDA receptor antagonist that is effective to increase transdermal flux of an opioid agonist in that compartment.

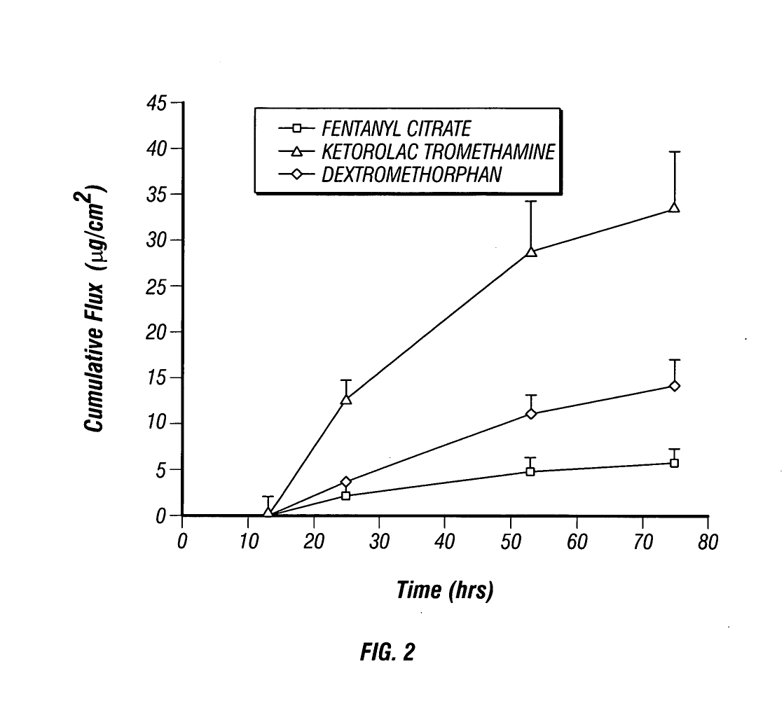

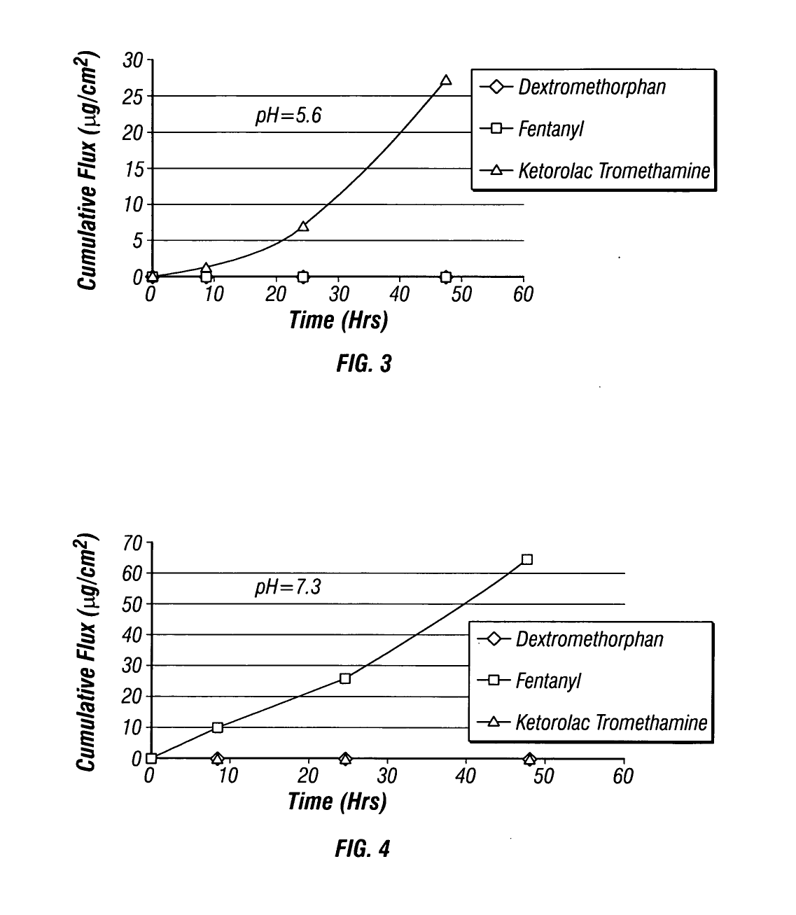

Placement of at least a portion of the NMDA receptor antagonist and the anti-inflammatory in separate compartments of a multi-compartment transdermal delivery patch provides significant benefits, as compared to the use of single compartment patches for the delivery of similar compositions. FIG. 2 is a plot illustrating the flux of fentanyl, ketorolac tromethamine and dextromethorphan from a single compartment of a transdermal delivery patch through human cadaver epidermis as a function of time, as described in Example 16 of U.S. application Ser. No. 11/097,878. FIG. 2 shows that a single compartment reservoir patch may be used to effectively deliver all three active ingredients through human skin. However, a formulation flexibility problem has now been discovered with such single compartment reservoir patch systems. In particular, it has been discovered that the delivery of desired dosages and the stability of the three active ingredients is undesirably sensitive to formulation pH, as demonstrated by the plots shown in FIGS. 3-5.

FIGS. 3-5 are plots illustrating the in vitro flux of fentanyl, ketorolac tromethamine and dextromethorphan through human cadaver epidermis as a function of time at pH values of 5.6, 7.3 and 8.6 using the Franz cell diffusion method generally described in Examples 12-15 of U.S. application Ser. No. 11/097,878. All three active ingredients were dissolved in the 80/20 ethanol/water solutions used to determine the in vitro flux, and thus the data are representative of flux from a single compartment of a transdermal delivery patch.

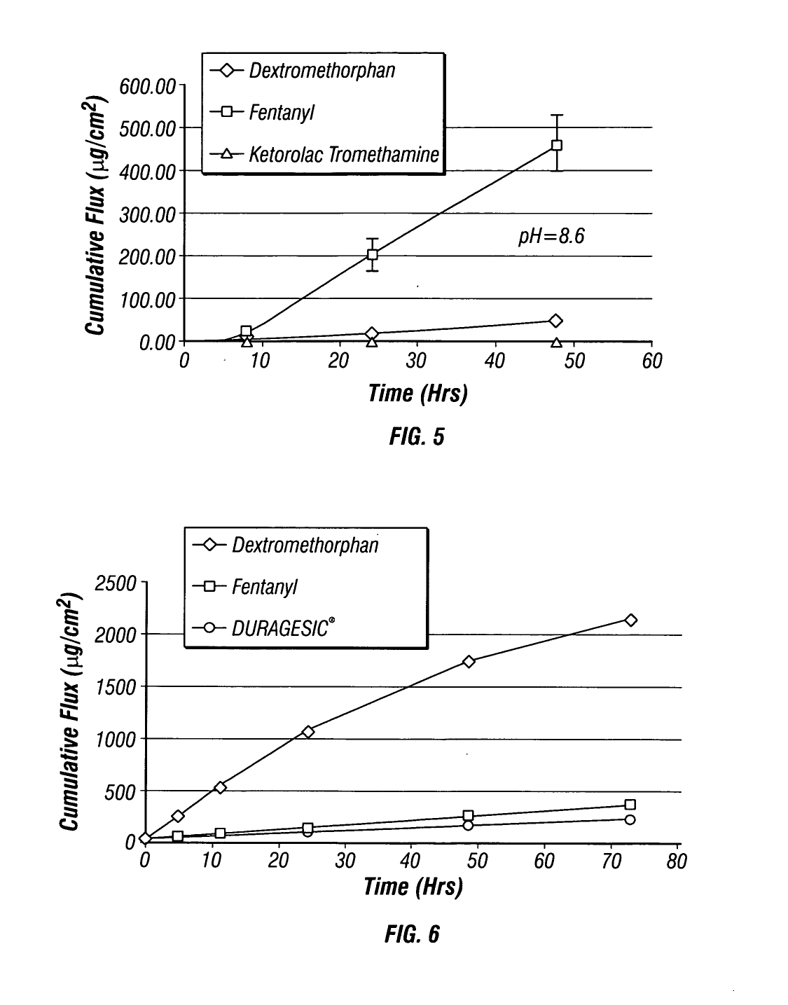

The plots shown in FIGS. 3-5 demonstrate the effect of pH on the transdermal delivery of clinically significant amounts of the active ingredients, under the experimental conditions employed. At a pH of 5.6, FIG. 3 shows that the flux of ketorolac tromethamine is clinically significant, whereas the fluxes of fentanyl and dextromethorphan are clinically insignificant. At a higher pH of 7.3, FIG. 4 shows that the flux of fentanyl has increased to a clinically significant level, whereas the flux of dextromethorphan remains clinically insignificant and the flux of ketorolac tromethamine has decreased to a clinically insignificant level. At a still higher pH of 8.6, FIG. 5 shows that the flux of fentanyl remains clinically significant and that the flux of dextromethorphan is higher than at a pH of 7.3 (see FIG. 4), but the flux of ketorolac tromethamine remains clinically insignificant.

It will be appreciated that routine experimentation may be used to identify formulations suitable for the transdermal delivery of clinically significant amounts of the active ingredients using single compartment patches. For example, as demonstrated in FIG. 2, ethanolic solutions of the three active ingredients are generally less sensitive to pH than the 80/20 ethanol/water solutions used to obtain the data shown in FIGS. 3-5. However, pH sensitivity remains a practical constraint on single compartment formulations. Because additional constraints such as cost, manufacturability, regulatory, shelf-life, consumer acceptance, etc. may also be imposed on the formulations in the course of commercialization and ultimate use by the consumer, it is desirable to eliminate or reduce the pH sensitivity problem.

The multi-compartment transdermal delivery patches described herein provide a solution to the pH sensitivity problem discovered by the inventors. In particular, placement of at least a portion of the NMDA receptor antagonist and the anti-inflammatory in separate compartments permits independent formulation of each to enhance skin-permeability. For example, the pH of one compartment may be adjusted to a basic pH, e.g., to facilitate transdermal delivery of an anti-inflammatory such as ketorolac tromethamine as illustrated in FIG. 3, and the pH of another compartment may be adjusted to an acidic pH, e.g., to facilitate transdermal delivery of an opioid agonist, NMDA receptor antagonist or mixture thereof as illustrated in FIGS. 4-5.

FIG. 6 is a plot illustrating the flux of fentanyl and dextromethorphan from a 80/20 ethanol/water solution containing both fentanyl (32.5 mg/mL) and dextromethorphan (saturated) at a pH of 9.6 through human cadaver epidermis as a function of time, as compared to the flux of fentanyl from a commercially available DURAGESIC® fentanyl patch. The concentration of fentanyl in the fentanyl/dextromethorphan mixture was 32.5 mg/mL (slightly higher than the 25 mg/mL fentanyl concentration in the DURAGESIC® fentanyl patch). The fluxes of fentanyl and dextromethorphan from the mixture are representative of the flux from an opioid agonist/NMDA receptor antagonist mixture in one compartment of a multi-compartment transdermal delivery patch. FIG. 6 shows that the fentanyl flux from the fentanyl/dextromethorphan mixture was comparable to the fentanyl flux from the commercial DURAGESIC® fentanyl patch.

### EXAMPLE 1

An empty two-compartment reservoir patch was obtained from a commercial source. The empty patch had a backing layer (outer layer exposed to environment), a reservoir layer (with two compartments, each having a volume of about 0.2 mL), a membrane layer having a surface area of about 14 cm² (about 7 cm² for each compartment, to control the flow of the active ingredients from the reservoir to the skin), a silicon adhesive layer (to adhere the membrane layer to the skin) and a protective liner (to be peeled from the adhesive layer prior to placement on the skin). The empty patch was also equipped with injection ports for each of the compartments to permit the active ingredients to be injected into the reservoir layer.

A first pharmaceutical composition was prepared in a laminar flow glove box using sterile technique as follows: A first solution having a total weight of about 10 grams was prepared by stirring together about 0.1 grams hydroxyethylcellulose (thickening agent), about 0.325 grams fentanyl (opioid agonist), about 0.910 grams dextromethorphan (NMDA receptor antagonist), and about 12.1 mL ethanol (USP). A small amount of base (sodium hydroxide) was added to adjust the pH to about 9.5. The total volume of the resulting viscous solution was about 12.5 mL. A portion of the viscous solution was drawn into a 5 mL syringe using a 16 gauge needle. The 16 gauge needle on the 5 mL syringe was detached and replaced with a 21 gauge needle. The 21 gauge needle was inserted into the injection port of the empty patch and about 0.2 mL of the viscous solution was injected into the first compartment of the two-compartment reservoir patch. The needle was gently removed from the port, and the patch was gently massaged to expel remaining air from the reservoir. The port was then sealed.

A second pharmaceutical composition was prepared in a laminar flow glove box using sterile technique as follows: A second solution having a total weight of about 10 grams was prepared by stirring together about 0.1 grams hydroxyethylcellulose (thickening agent), about 0.850 grams ketorolac tromethamine (anti-inflammatory) and about 11.7 mL ethanol (USP). A small amount of hydrochloric acid was added to adjust the pH to about 4.5. The total volume of the resulting viscous solution was about 12.5 mL. A portion of the viscous solution was drawn into a 5 mL syringe using a 16 gauge needle. The 16 gauge needle on the 5 mL syringe was detached and replaced with a 21 gauge needle. The 21 gauge needle was inserted into the injection port of the empty patch and 0.2 mL of the viscous solution was injected into the second compartment of the two-compartment reservoir patch. The needle was gently removed from the port, and the patch was gently massaged to expel remaining air from the reservoir. The port was then sealed.

The sealed first compartment of the resulting two-compartment transdermal delivery patch contained about 6.5 mg of fentanyl (about 32.5 mg/mL) and about 19.4 mg of dextromethorphan (about 97 mg/mL) at a pH of about 9.6, and the sealed second compartment contained about 17 mg of ketorolac tromethamine (about 85 mg/mL) at a pH of about 4.5.

### EXAMPLES 2-11

A series of two-compartment transdermal delivery patches are prepared in the general manner described in EXAMPLE 1, except that the sizes of the patches and the amounts and types of opioid agonist, NMDA receptor antagonist, and anti-inflammatory are varied as shown in TABLE 1. The surface areas and volumes of each compartment were approximately equal (each about half of total patch size).

TABLE 1

| | | | Compartment 1 | | |
|---|---|---|---|---|---|
| | Patch | | | NMDA Receptor | Compartment 2 |
| No. | Size | | Opioid Agonist | Antagonist | Anti-Inflammatory |
| 2 | 14 | cm² | 1.96 mg Fentanyl | 12 mg Dextromethorphan | 10 mg Ketorolac |
| | 0.4 | mL | 0.196 mg Sufentanil | | |
| 3 | 28 | cm² | 5.88 mg Fentanyl | 36 mg Dextromethorphan | 30 mg Ketorolac |
| | 1.2 | mL | 0.588 mg Sufentanil | | |
| 4 | 42 | cm² | 8.82 mg Fentanyl | 54 mg Dextromethorphan | 45 mg Ketorolac |
| | 1.8 | mL | 0.882 mg Sufentanil | | |
| 5 | 56 | cm² | 11.76 mg Fentanyl | 72 mg Dextromethorphan | 60 mg Ketorolac |
| | 2.4 | mL | 1.176 mg Sufentanil | | |
| 6 | 70 | cm² | 14.7 mg Fentanyl | 90 mg Dextromethorphan | 75 mg Ketorolac |
| | 3.0 | mL | 1.47 mg Sufentanil | | |
| 7 | 14 | cm² | 1.96 mg Fentanyl | 15 mg Amantadine | 10 mg Ketorolac |
| | 0.4 | mL | 0.196 mg Sufentanil | | |
| 8 | 14 | cm² | 1.96 mg Fentanyl | 30 mg Amitriptyline | 10 mg Ketorolac |
| | 0.4 | mL | 0.196 mg Sufentanil | | |
| 9 | 14 | cm² | 1.96 mg Fentanyl | 45 mg Methadone | 10 mg Ketorolac |
| | 0.4 | mL | 0.196 mg Sufentanil | | |
| 10 | 14 | cm² | 1.96 mg Fentanyl | 12 mg Dextromethorphan | 2 mg Dexamethasone |
| | 0.4 | mL | 0.196 mg Sufentanil | | |
| 11 | 14 | cm² | 1.96 mg Fentanyl | 12 mg Dextromethorphan | 1 mg Betamethasone |
| | 0.4 | mL | 0.196 mg Sufentanil | | |

### EXAMPLE 12

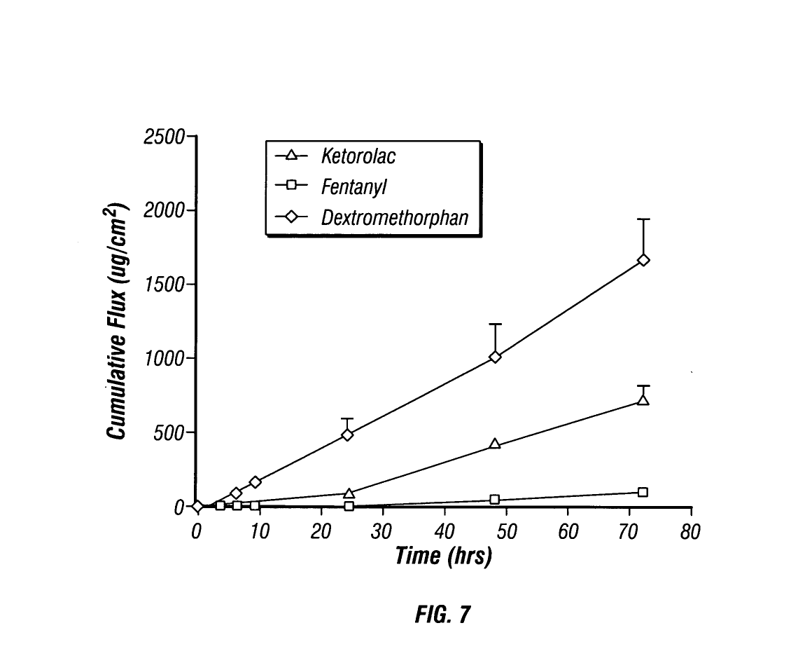

An empty two-compartment reservoir patch was obtained and loaded with fentanyl, dextromethorphan and ketorolac tromethamine as described in EXAMPLE 1. The patch was applied to human cadaver epidermis and in vitro flux from each compartment was measured using a Franz cell having two donor chambers and a single receiver chamber in accordance with the diffusion method generally described in Examples 12-15 of U.S. application Ser. No. 11/097,878. The resulting flux data is shown in Table 2 below and plotted as a function of time in FIG. 7. The data demonstrates that a multi-compartment reservoir patch may be used to effectively deliver all three active ingredients through human skin. In particular, the data shows that the multi-compartment patches described herein can be used to deliver all three active ingredients transdermally in clinically significant amounts and at higher fluxes than using a single compartment patch (compare to FIG. 2, note difference in scale). The relatively high levels of dextromethorphan provide a significant clinical advantage by reducing or preventing opioid tolerance.

TABLE 2

| | Cumulative Flux (μg/cm²) | | | | | | |
|---|---|---|---|---|---|---|---|
| | 0 hr. | 3 hr. | 6 hr. | 9 hr. | 24 hr. | 48 hr. | 72 hr. |
| Ketorolac | 0 | 24 | 7 | 11 | 100 | 412 | 726 |
| Fentanyl | 0 | 1 | 2 | 4 | 11 | 41 | 89 |
| Dextromethorphan | 0 | 37 | 112 | 174 | 487 | 1022 | 1682 |

Although the foregoing invention has been described in terms of certain preferred embodiments, other embodiments will become apparent to those of ordinary skill in the art in view of the disclosure herein. Accordingly, the invention is not intended to be limited by the recitation of preferred embodiments, but is intended to be defined solely by reference to the appended claims.

What is claimed is:

1. A multi-compartment transdermal delivery patch, comprising pharmaceutically effective amounts of skin-permeable forms of an opioid agonist, an N-methyl-D-aspartate (NMDA) receptor antagonist different from the opioid agonist, and an anti-inflammatory different from the opioid agonist and different from the NMDA receptor antagonist; wherein at least a portion of the NMDA receptor antagonist and the anti-inflammatory are in separate compartments.

2. The multi-compartment transdermal delivery patch of claim 1, comprising:
a first compartment containing a first pharmaceutical composition, the first pharmaceutical composition comprising the opioid agonist and the NMDA receptor antagonist; and
a second compartment containing a second pharmaceutical composition, the second pharmaceutical composition comprising the anti-inflammatory.

3. The multi-compartment transdermal delivery patch of claim 2, wherein the first pharmaceutical composition has a pH in the range of about 8 to about 12.

4. The multi-compartment transdermal delivery patch of claim 2, wherein the first pharmaceutical composition has a pH in the range of about 9 to about 11.

5. The multi-compartment transdermal delivery patch of claim 2, wherein the second pharmaceutical composition has a pH in the range of about 3 to about 6.

6. The multi-compartment transdermal delivery patch of claim 2, wherein the second pharmaceutical composition has a pH in the range of about 4 to about 5.

7. The multi-compartment transdermal delivery patch of claim 2, wherein the first pharmaceutical composition has a pH in the range of about 8 to about 12 and the second pharmaceutical composition has a pH in the range of about 3 to about 6.

8. The multi-compartment transdermal delivery patch of claim 2, wherein the opioid agonist is selected from fentanyl, sulfentanil, hydromorphone, oxymorphone, hydrocodone, oxycodone, morphine, methadone, meperidine, ketamine, and propoxyphene.

9. The multi-compartment transdermal delivery patch of claim 8, wherein the opioid agonist is selected from fentanyl and sulfentanil.

10. The multi-compartment transdermal delivery patch of claim 9, wherein the opioid agonist is in the form of a pharmaceutically acceptable salt.

11. The multi-compartment transdermal delivery patch of claim 2, wherein the NMDA receptor antagonist is selected from dextromethorphan, amitriptyline, amantadine, ketamine, methadone, and D,L-2-amino-5-phosphono valeric acid.

12. The multi-compartment transdermal delivery patch of claim 2, wherein the NMDA receptor antagonist is in the form of a pharmaceutically acceptable salt.

13. The multi-compartment transdermal delivery patch of claim 2, wherein the anti-inflammatory is in the form of a pharmaceutically acceptable salt.

14. The multi-compartment transdermal delivery patch of claim 2, wherein the anti-inflammatory is a nonsteroidal anti-inflammatory.

15. The multi-compartment transdermal delivery patch of claim 14, wherein the nonsteroidal anti-inflammatory is selected from ketorolac, ibuprofen, nabumetone, diclofenac, etodolac, and piroxicam.

16. The multi-compartment transdermal delivery patch of claim 2, wherein the opioid agonist is selected from fentanyl and sufentanil, the NMDA receptor antagonist is dextromethorphan, and the anti-inflammatory is ketorolac.

17. The multi-compartment transdermal delivery patch of claim 2, wherein both the first pharmaceutical composition and the second pharmaceutical composition are free of a pharmaceutically effective amount of an α3β4 nicotinic receptor antagonist.

18. The multi-compartment transdermal delivery patch of claim 2, wherein at least of one of the first pharmaceutical composition and the second pharmaceutical composition further comprises a skin permeation enhancer.

19. The multi-compartment transdermal delivery patch of claim 18, wherein the skin permeation enhancer comprises linoleic acid.

20. The multi-compartment transdermal delivery patch of claim 19, wherein the opioid agonist is selected from fentanyl and sufentanil, the NMDA receptor antagonist is dextromethorphan, the anti-inflammatory is ketorolac, and the second pharmaceutical composition comprises the linoleic acid.

21. A method for treating pain, comprising applying the multi-compartment transdermal delivery patch of claim 2 to a human suffering from pain for a period of time effective to at least partially relieve the pain.

22. The method of claim 21, wherein the pain is chronic pain.

23. The method of claim 21, wherein the opioid agonist is selected from fentanyl and sufentanil, the NMDA receptor antagonist is dextromethorphan, the anti-inflammatory is ketorolac, and the second pharmaceutical composition comprises the linoleic acid.

24. The method of claim 21, wherein the first pharmaceutical composition comprises an amount of NMDA receptor antagonist that is effective to increase transdermal flux of the opioid agonist.

25. The multi-compartment transdermal delivery patch of claim 1, comprising:
a first compartment containing the opioid agonist;
a second compartment containing the NMDA receptor antagonist; and
a third compartment containing the anti-inflammatory.

26. The multi-compartment transdermal delivery patch of claim 25, wherein the opioid agonist is at a pH in the range of about 8 to about 12.

27. The multi-compartment transdermal delivery patch of claim 25, wherein the NMDA receptor antagonist is at a pH in the range of about 8 to about 12.

28. The multi-compartment transdermal delivery patch of claim 25, wherein the anti-inflammatory is at a pH in the range of about 3 to about 6.

29. The multi-compartment transdermal delivery patch of claim 25, wherein the opioid agonist is selected from fentanyl and sufentanil, the NMDA receptor antagonist is dextromethorphan, and the anti-inflammatory is ketorolac.

30. A method for treating pain, comprising applying the multi-compartment transdermal delivery patch of claim 25 to a human suffering from pain for a period of time effective to at least partially relieve the pain.

31. The method of claim 30, wherein the pain is chronic pain.
